(19) United States
(12) Patent Application Publication
(10) Pub. No.: US 2011/0021455 A1
(43) Pub. Date: Jan. 27, 2011

(54) STERILISED SUCRALFATE GEL

(75) Inventors: Sophie Chesnoy, Lille (FR); Marlène Delaunois, Cessales (FR); Sandrine Rouge, Pins-Justaret (FR)

Correspondence Address: BIRCH STEWART KOLASCH & BIRCH, PO BOX 747, FALLS CHURCH, VA, 22040-0747, US

(73) Assignee: PIERRE FABRE DERMO-COSMETIQUE, BOULOGNE-BILLANCOURT (FR)

(21) Appl. No.: 12/919,238

(22) PCT Filed: Feb. 26, 2009
(86) PCT No.: PCT/EP09/52251

§ 371 Date: Aug. 25, 2010

(30) Foreign Application Priority Data
Feb. 26, 2008 (FR) 0851206

(51) Int. Cl. A61K 31/7016; A61P 17/02

(52) U.S. Cl. 514/53

(57) ABSTRACT

The present invention relates to an autoclave-sterilized gel comprising sucralfate and at least 15% by weight of a humectant, relative to the total weight of gel, and having a pH of greater than or equal to 6, more particularly for use in a skin healing treatment by topical application.

The present invention relates to an autoclave-sterilised sucralfate gel, as well as a pharmaceutical or cosmetological composition comprising this gel, such as a sterile liquid dressing, and its use for cicatrisation of the skin.

There are currently numerous commercially available liquid dressings which are generally used to enable cicatrisation of small wounds of the face or body.

In the field of cicatrisation of more substantial wounds, particularly eschars and ulcers, various liquid dressings are also known, including hydrogel-based dressings, the influence of which on epithelisation of wounds is known (Acta Derm. Venereol. 1998, 78, pp. 119-122).

Humectant substances such as glycerine, propylene glycol or sorbitol, as well as compounds with healing action such as sucralfate for example can be added to gelling substances used such as carboxymethyl cellulose, xanthan or guar gums or alginates, as well as to water, which are the essential constituents of these gels.

However, these dressings are rarely sterile. Sterilisation does however avoid using often-irritating preservatives such as sorbic acid. And eschars and ulcers are the consequence of complex pathologies in fragile patients who are therefore sensitive to any risk of allergy and infection. Hospitals consequently prefer using sterile liquid dressings.

However, sterilisation of such gels is not evident. The selected process must be done such that no active compound is commonly during or after the sterilisation procedure, and such that the different compounds do not interact.

Sucralfate is an aluminium salt of sucrose octasulfate. It is known for improving cicatrisation and is commonly used in the treatment of gastric and duodenal ulcers and even burns (Burns 2001, 27, pp. 465-469).

Some patents describe topical use of sucralfate in the form of gel in healing.

FR 2 646 604 describes a sucralfate composition in the form of gel. The sucralfate solution is acidified at pH close to 4.5 to produce a stable composition having anti-inflammatory and healing properties. However, this patent does not describe sterile sucralfate gel.

EP 402 933 describes a humid, solid or liquid sucralfate gel obtained from particulate sucralfate, the average diameter of the particles having to be less than 6 μm and their specific surface greater than 200 m²/g, and capable of being linked to an active ingredient of amikacine type. According to the examples, this gel can be sterilised by gamma rays.

However, the indications given in this patent have not allowed to reproduce humid sucralfate gel and only a pasty composition was obtained and not a gel. Because of this, no sterile gel was reproduced via the process described in this patent.

The inventors discovered that the autoclave is the sole known means of sterilisation compatible with sucralfate.

In fact, after various tests, it seemed that sucralfate, both in the form of powder and gel, is unstable during sterilisation by gamma rays since the composition yellows. Similarly, during sterilisation with ethylene oxide, substantial adsorption of the latter on sucralfate requires a long and costly desorption phase to avoid the presence of toxic residue unfit for the desired use (pharmaceutical or cosmetological) of sucralfate.

Also, the inventors noted that the pH of the gel to be sterilised was an important factor since gels having pH lower than 6 do not exhibit good stability following sterilisation (up to 75% degradation of the sucralfate used), whereas gels of pH above or equal to 6 remain stable following sterilisation (less than 10% degradation of the sucralfate used).

The aim of the present invention therefore is a gel sterilised by autoclave comprising sucralfate and at least 15% by weight of a humectant relative to the total weight of the gel, and a pH above or equal to 6.

In fact, it was noted that with such pH and humectant dosage conditions, a stable sucralfate gel was produced which could support thermal sterilisation by autoclave. So, such a sucralfate gel remains stable during and after sterilisation.

This is probably because, since the pH and the quantity of humectant are regulated, solubilisation of the sucralfate is limited and this must protect it from degradation at high heat.

Advantageously, the pH of the gel of the invention will be between 6 and 7, and preferably between 6 and 6.8.

In a particular embodiment, the gel of the invention comprises from 5 to 15%, and preferably 10% by weight of sucralfate relative to the total weight of the gel to allow better efficacy on cicatrisation.

Advantageously, the humectant could be selected from sorbitol, glycerol and propylene glycol. Sorbitol will preferably be selected since it is less irritating and sensitizing.

Advantageously, the gel of the invention will contain from 15 to 25%, preferably from 15 to 20%, and more preferably from 15 to 18% of humectant.

Also, glycerol will be discouraged for diabetics.

Advantageously, the gel of the invention will be hydrogel and therefore could comprise at least 60% and preferably at least 69% by weight of water, relative to the total weight of gel.

A gelling agent could also be added to the gel of the invention to produce a composition in the form of gel, and especially in a quantity of 0.5% to 6% by weight, relative to the total weight of the gel.

The gelling agent could especially be selected from hydroxyethyl cellulose, hydroxypropyl cellulose, hydroxypropyl methylcellulose and silican, and will preferably be hydroxyethyl cellulose or silican.

The silican used could be in particular colloidal silican such as Aerosil® v200 sold by Degussa.

Advantageously, the gelling agent will be hydroxyethyl cellulose.

To get a gel with the desired pH, the gel of the invention could also comprise a pharmaceutically acceptable acid or base.

“Pharmaceutically acceptable acid” is understood in terms of the present invention as any non-toxic acid, including organic and inorganic acids, which acids include hydrochloric acid, bromhydric acid, sulphuric acid, nitric acid, phosphoric acid, acetic acid, benzenesulfonic acid, benzoic acid, camphresulfonic acid, citric acid, ethane-sulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, mandelic acid, methanesulfonic acid, muconic acid, 2-naphtalenesulfonic acid, propionic acid, salicylic acid, succinic acid, dibenzol-L-tartaric acid, tartaric acid, p-toluenesulfonic acid, trimethylacetic acid and trifluoroacetic acid.

“Pharmaceutically acceptable base” is understood in terms of the present invention as any non-toxic base, including organic and inorganic acids.

Examples of inorganic base are bases forming for example ammonium salts or salts of alkaline or alkaline earth metals such as lithium, sodium, potassium, magnesium or even calcium, such as especially aluminium hydroxide, calcium hydroxide, potassium hydroxide, sodium carbonate or sodium hydroxide.

Examples of organic base are diethanolamine, ethanolamine, N-methylglucamine, triethanolamine, triethylamine or tromethamine.

According to a particular characteristic of the invention, the sterilised sucralfate gel contains no preservative, and in particular preservatives which might be absorbed systemically.

“Preservative” is understood especially as methyl parahydroxybenzoate, propyl parahydroxybenzoate, butyl parahydroxybenzoate, phenoxyethanol, chlorphenesine or sorbic acid.

According to a particular embodiment, sterilisation by autoclave of the gel of the invention could be done at a temperature of 121° C. over a period of 15 minutes in conditions familiar to the person skilled in the art. Such sterilisation is carried out in saturating water vapour.

The aim of the present invention is also a pharmaceutical or cosmetologic composition comprising sterilised sucralfate gel such as defined hereinabove for topical application.

In particular, such a composition could be a sterile liquid dressing.

Another aim of the present invention is the use of a sterilised sucralfate gel such as defined hereinabove for the preparation of a pharmaceutical or cosmetologic composition intended for a healing treatment of the skin, and especially eschars and/or ulcers.

### EXAMPLES

### 1. Influence of the Sucralfate Dose

Two hydrogels comprising respectively 5 and 10% of sucralfate by weight and a pH equal to 4 were prepared with the following composition:

| Composition | Hydrogel 1 | Hydrogel 2 |
|---|---|---|
| Sucralfate | 5 g | 10 g |
| Aerosil ® V200 | 10.5 g | 9 g |
| Sorbitol | 16.45 g | 10.5 g |
| Total water | qs 100 g | qs 100 g |

(qs = sufficient quantity for)

These two hydrogels were sterilised in an autoclave at 121° C. for 15 min and the following results were obtained:

| Analytical Tests | Standards | Hydrogel 1 | Hydrogel 2 |
|---|---|---|---|
| pH before | 4.0 ± 0.5 | 3.95 | 4.1 |
| autoclave | | | |
| pH after | 4.0 ± 0.5 | 3.62 | 3.75 |
| autoclave | | | |
| Sucralfate | 1.4 to 1.5% | 1.6%* | 10.0% |
| dosage before | or | | |
| sterilisation | 9.0 to 11.0% | | |
| Sucralfate | 1.4 to 1.5% | 0.4%* | 6.4% |
| dosage after | or | | |
| sterilisation | 9.0 to 11.0% | | |

*In this case, sucralfate dosage corresponds to sucrose octasulfate dosage which comprises sucralfate at a level of 30 to 38% in general.

It is therefore evident that degradation of sucralfate for compositions formulated at pH=4 rises to 75% for the first formulation comprising 5% of sucralfate, whereas it is only 36% for the second formulation comprising 10% of sucralfate.

So, when the sucralfate content is lower in the composition, increasing its solubilisation in aqueous medium, its degradation is also higher.

### 2. Influence of pH

Two novel hydrogels comprising 10% of sucralfate were prepared at a higher pH equal to 6 with the following composition:

| Composition | Hydrogel 3 | Hydrogel 4 |
|---|---|---|
| Sucralfate | 10 g | 10 g |
| Hydroxyethyl cellulose | 4 g | 4 g |
| Sodium hydroxide | 0.276 g | 0.276 g |
| Sorbitol | 16.45 g | 10.45 g |
| Total water | qs 100 g | qs 100 g |

These two hydrogels were then sterilised in an autoclave at 121° C. for 15 min and the following results were obtained:

| Analytical Tests | Standards | Hydrogel 3 | Hydrogel 4 |
|---|---|---|---|
| pH before | 6.0 ± 0.5 | 6.08 | 6.2 |
| autoclave | | | |
| pH after | 6.0 ± 0.5 | 5.42 | 5.5 |
| autoclave | | | |
| Sucralfate | 9.0 to | 9.44% | 9.52% |
| dosage before | 11.0% | | |
| sterilisation | | | |
| Sucralfate | 9.0 to | 9.25% | 8.68% |
| dosage after | 11.0% | | |
| sterilisation | | | |

It is therefore evident that stability of sucralfate during sterilisation is improved with formulations at higher pH since degradation of sucralfate in hydrogels 3 and 4 is less than 10% in both cases.

Also, it is noted that stability is slightly better when the sorbitol (humectant) content in the composition is greater.

### 3. Influence of the Humectant Dose

Studies of accelerated stability were conducted over 4 months at 40° C. on the preceding hydrogels 3 and 4 and led to the following results:

| Analytical | Standards | Hydrogel 3 | Hydrogel 4 |
|---|---|---|---|
| Tests | | | |
| Dosage after | 9.0 to | 9.21% | 7.35% |
| 4 months at 40° C. | 11.0% | | |

It is therefore noted that hydrogel having a sorbitol (humectant) content of more than 15% remains stable over time, contrary to hydrogel having a sorbitol content of 10%.

1. A gel sterilised by autoclave comprising sucralfate and at least 15% by weight of a humectant, relative to the total weight of the gel, and of a pH greater than or equal to 6.

2. The gel as claimed in claim 1, wherein it comprises from 5 to 15%, and preferably 10% by weight of sucralfate, relative to the total weight of the gel.

3. The gel as claimed in any one of claims 1 and 2, wherein the humectant is selected from sorbitol, glycerol and propylene glycol, and is preferably sorbitol.

4. The gel as claimed in claim 1, wherein it comprises at least 60% of water, relative to the total weight of the gel.

5. The gel as claimed in claim 1, further comprising a gelling agent.

6. The gel as claimed in claim 5, wherein it comprises from 0.5% to 6% by weight of the gelling agent relative to the total weight of the gel.

7. The gel as claimed in claim 5, wherein the gelling agent is selected from the group consisting of hydroxyethyl cellulose, hydroxypropyl cellulose, hydroxypropyl methyl cellulose and silican.

8. The gel as claimed in claim 1, wherein it comprises further comprising a pharmaceutically acceptable acid or base.

9. The gel as claimed in claim 1, wherein it contains no preservative.

10. The gel as claimed in claim 1, wherein sterilisation via autoclave is carried out at a temperature of 121° C. over a period of 15 minutes.

11. A pharmaceutical or cosmetologic composition comprising a gel as claimed in claim 1 for topical application.

12. The composition as claimed in claim 11, which is sterile liquid dressing.

13. A method of healing treatment of a condition of the skin, which comprises administering to patient in need thereof, a pharmaceutical or cosmetologic composition comprising the gel as claimed in claim 1.

14. The gel as claimed in claim 4, wherein it comprises at least 69% by weight of water, relative to the total weight of the gel.

15. The gel as claimed in claim 7, wherein the gelling agent is hydroxyethyl cellulose or silican.

16. The method of treatment of claim 13, wherein the healing treatment is a healing treatment of eschars and/or ulcers.

17. The method of treatment of claim 13, wherein said composition is topically administered.

18. The gel as claimed in claim 2, wherein it comprises 10% by weight of sucralfate, relative to the total weight of the gel.

19. The gel as claimed in claim 3, wherein the humectant is sorbitol.
